(19) United States
(12) Patent Application Publication
(10) Pub. No.: US 2018/0311482 A1
(43) Pub. Date: Nov. 1, 2018

(54) DRUG COATED BALLOON CATHETER ARTERIOVENOUS SHUNT

(71) Applicant: ACOTEC SCIENTIFIC CO. LTD., Beijing (CN)

(72) Inventor: Silvio SCHAFFNER, Beijing (CN)

(21) Appl. No.: 15/770,580

(22) PCT Filed: Oct. 30, 2015
(86) PCT No.: PCT/IB2015/002090

§ 371 Date: Apr. 24, 2018

(51) Int. Cl. A61M 25/10

(52) U.S. Cl. CPC A61M 25/104; A61M 25/1002; A61M 2025/1084; A61M 2025/1075; A61M 2025/1079; A61M 2025/105

(57) ABSTRACT

A drug coated balloon (DCB) catheter (4) comprising a connector (8), a shaft (20) extending from a proximal end (24) to a distal end (28) along an axial direction (X-X) and having a guidewire lumen (32) and an inflation lumen (36), the shaft (20) being connected to the connector (8) on said proximal end (24) and provided with an inflatable balloon (48), fluidically connected with said inflation lumen (36) in order to be selectively inflated and/or deflated. Advantageously the balloon (48) is conical and axis-symmetrical around a prevalent extension axis (S-S), wherein the balloon (48) has an increasing diameter (60), moving from a first end (52) to a second end (56), said diameter (60) being measured on a section plane perpendicular to said prevalent extension axis (S-S), wherein an external surface (64) of the balloon (48) is coated with a drug for treating restenosis.

### DESCRIPTION

The present invention relates to a balloon catheter for arteriovenous (AV) shunt.

In particular, AV-shunt patients have high calcified lesions and very high restenosis rate. Therefore, they need dilation of the artery several times with high pressure.

It is known in the art to realize the dilation of a restenosis by means of percutaneous transluminal angioplasty (PTA) balloons.

It is also known in the art to use drug eluted balloon (DEB) catheters or drug coated balloon (DCB) catheters wherein the drug helps the local treatment of the lesion or restenosis, from the inner side of the vessel wall.

Such known catheters have some drawbacks: in fact DEB PTA balloons have small diameters and low inflating (and burst) pressures. In addition, they do not have indication of specific dimensions.

Therefore, known PTA balloons are not able to dilate the restenosis efficiently, in particular for AV-shunt restenosis.

The purpose of the present invention is that of providing a catheter which overcomes the drawbacks mentioned with reference to the prior art; in other words a catheter for AV shunt which can easily reach the target lesion, completely dilate the restenosis and significantly reduce the restenosis rate.

Such aim is reached by a catheter according to claim 1.

Other embodiments of the catheter according to the invention are described in the subsequent claims.

Further characteristics and advantages of the present invention will be more clearly comprehensible from the description given below of its preferred and non-limiting embodiments, wherein:

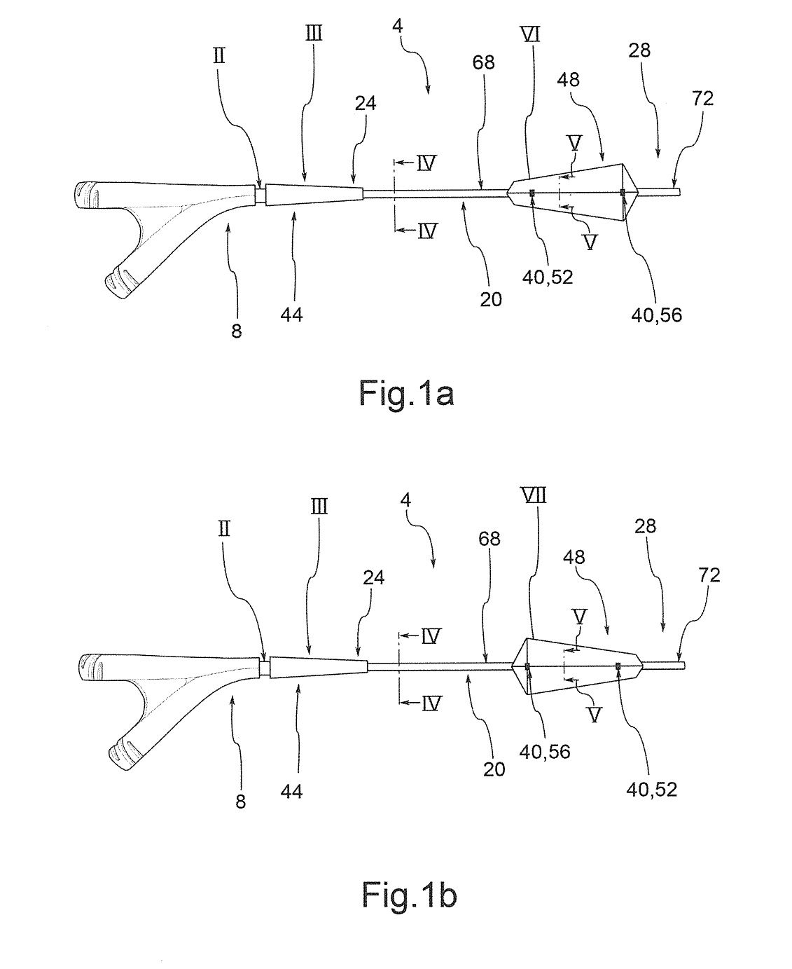

FIG. 1a shows a perspective view of a drug coated balloon (DCB) catheter according to an embodiment of the present invention;

FIG. 1b shows a perspective view of a drug coated balloon (DOB) catheter according to another embodiment of the present invention;

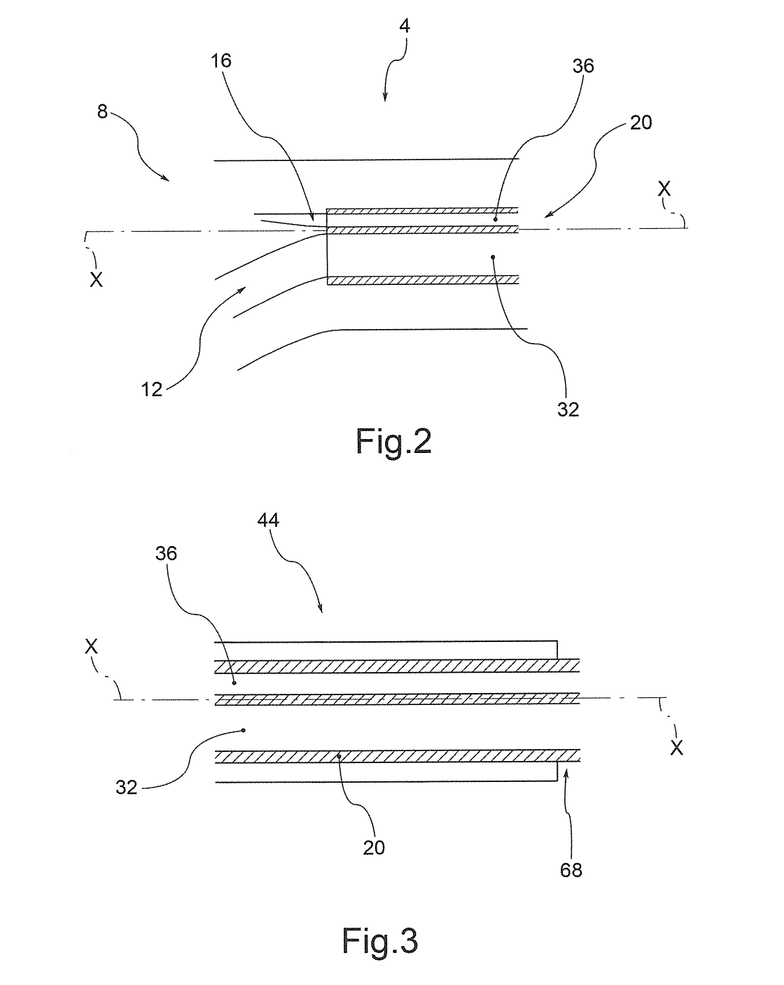

FIG. 2 shows a longitudinal section view of particular II of the catheter of FIGS. 1a-1b;

FIG. 3 shows a longitudinal section view of particular III of the catheter of FIGS. 1a-1b;

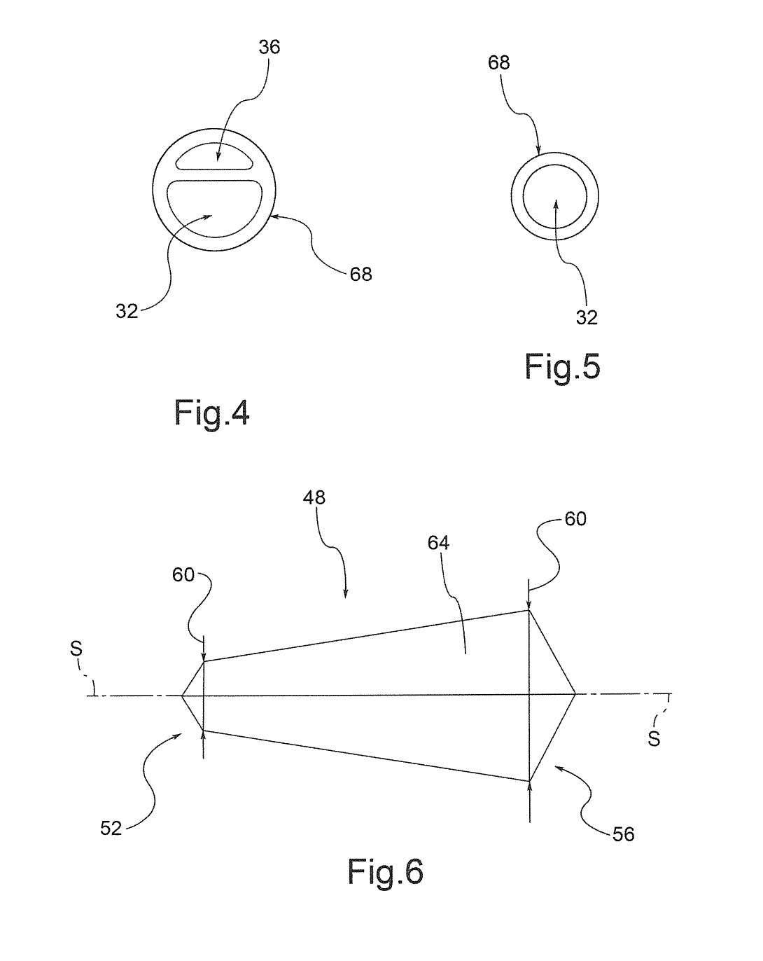

FIG. 4 FIG. 4 shows a section view of the catheter of FIG. 1a, taken along section line IV-IV shown in FIGS. 1a-1b;

FIG. 5 shows a section view of the catheter of FIGS. 1a-1b, taken along section line V-V shown in FIGS. 1a-1b;

FIG. 6 is an enlarged view of particular VI of FIGS. 1a-1b.

The elements or parts of elements common to the embodiments described below will be indicated using the same reference numerals.

With reference to the aforementioned figures, reference numeral 4 globally denotes a catheter, in particular, a drug coated balloon (DCB) catheter for releasing drug on a target lesion inside a blood vessel and for dilate a stenosis of a blood vessel, in particular in AV-shunt patients.

The catheter 4 comprises a connector 8 comprising a guide wire channel 12 and an inflation channel 16.

The guide wire channel 12 is useful for housing a guide wire for the guide and the insertion of the catheter 4 in a predetermined vessel.

Moreover, the inflation channel 16 is useful in order to send a pressure fluid to the distal end of the shaft of the catheter. For example the pressure fluid may be gas or a liquid. Moreover, the inflation channel may be used as a perfusion channel, for example, of a contrast liquid.

The catheter 4 comprises a shaft 20 extending from a proximal end 24 to a distal end 28 along an axial direction X-X.

Moreover, the shaft 20 have a guide wire lumen 32 and an inflation lumen 36.

Preferably, said guidewire lumen 32 is between 0.010 inches to 0.037 inches.

According to a possible embodiment, the catheter according to the present invention has at least a dual-lumen construction, comprising at least two lumens comprising a guidewire lumen 32 and an inflation lumen 36.

According to a possible embodiment, said guidewire lumen 32 and inflation lumen 36 are not coaxial each other. For example said guidewire lumen 32 and inflation lumen 36 are separated by an internal sept 44. For example said internal sept 44 delimits, at least partly, a wall of each of the separated lumens 32,36.

Anyway, the catheter 4 may have further lumens for different purposes.

The shaft 20 is connected to the connector 8 on said proximal end 24.

In particular, the shaft 20 is connected, at its proximal end 24, to the connector 8 so that said guide wire lumen 32 and inflation lumen 36 are mechanically and fluidically connected with said guide wire channel 12 and inflation channel 16 of the connector 8, respectively.

According to an embodiment of the present invention, the shaft 20 and the connector 8 are made up of a polymer material, such as Polyamide, Pebax, Polycarbonate and similar.

According to the invention, the shaft is provided with an inflatable balloon 48, which is fluidically connected with said inflation lumen 36 in order to be selectively inflated and/or deflated.

According to one embodiment, the balloon 48 is provided to a couple of markers 40, positioned in correspondence of a first end 52 and a second end 56 of the balloon 48, defining the axial length of the balloon itself. In particular, the balloon extends along a prevalent extension S-S.

Advantageously, the balloon 48 is conical and axis-symmetrical around said prevalent extension axis S-S.

The balloon 48 has an increasing diameter 60, moving from the first end 52 to the second end 56, said diameter being measured on a section plane perpendicular to said prevalent extension axis S-S. According to an embodiment said first end 52 faces the proximal end 24 and said second end 56 faces the distal end 28; according to another embodiment the first end 52 faces the distal end 28 and said second end 56 faces the proximal end 24.

Preferably, the diameter 80 of the balloon 48 continuously increases moving from the first end 52 to the second end 56.

According to a possible embodiment, the diameter 80 at the first end 52 is 6 mm.

According to a possible embodiment, the diameter 80 at the second end is 7 mm.

According to further embodiments, the diameter 80 at the second end is 8 mm or 9 mm.

According to an embodiment, the length of the balloon 48 or axial distance from the first end 52 to the second end 56 of the balloon 48 is between 30 mm and 60 mm.

Preferably, said length of the balloon 48 is about 45 mm.

Preferably, said conical balloon 48 is configured so as to have a nominal or working pressure between 12 to 16 bar.

Preferably, said conical balloon 48 is configured so as to have a burst pressure above 25 bar, preferably 30 to 40 bar. According to a possible embodiment the balloon 48 has an increased wall thickness. For example such a thickness is comprised between 0.01 mm and 0.2 mm; preferably such thickness is about double or triple than prior art balloons.

Such an increased wall thickness can be obtained by a single thicker sheath delimiting the balloon surface or by the juxtaposition of two balloons (so called double balloon construction). According to another possible embodiment, the balloon is stiffened by reinforcing fibers.

Advantageously, an external surface 64 of the balloon 48 is coated with a drug for treating restenosis.

Such a drug can be delivered by the balloon 48 on a target lesion or stenosis to be treated and enlarged.

The drug can be of any type, depending on the kind and positioning of lesion to be treated.

According to one embodiment, the drug formulation is a Paclitaxel solution.

Preferably, said drug on the external surface 64 of the balloon 48 has a concentration between 2 to 10 μg/mm².

According to an embodiment of the invention, an external wall 68 of the shaft 20, opposite to said lumens 32,36, is covered with a lubricant.

In this way, said external wall 68 which, during insertion of the catheter into the vessel, contacts the internal wall of the blood vessel or the internal wall of an introducer, sheath, guiding catheter or any other accessory used for intervention, is lubricated and can easily slip into the vessel itself.

According to the invention, the external wall 68 of the shaft 20 is covered with a lubricant except for said inflatable balloon 48.

In this way, the lubricant does not interfere with the action of the drug which has to be delivered on the target lesion.

The distal end 28 of the shaft 20 comprises a flexible tip 72 which aims the catheter to be cannulated inside blood vessels.

According to one embodiment, said flexible tip 72 is covered with lubricant too.

Advantageously, said guidewire lumen 32 is internally covered with a lubricant.

As it can be seen from the description, the catheter according to the invention makes it possible to overcome the drawbacks mentioned with reference to the prior art.

In particular, it is possible to treat efficiently an AV-shunt restenosis by means of a conical balloon which can reach the target lesion easily, and which can dilate the restenosis completely using high pressure so as to get the correct lumen.

Moreover, it is possible to treat the lesion by means of drug which is applied to the inner wall of the vessel to be dilated.

Moreover the specific geometry of the conical balloon implies that the external surface of the balloon is not constant along the axial extension of the balloon: in particular the external surface is smaller towards the portion with smaller diameter and it is bigger towards the portion with bigger diameter.

Therefore, in the catheter according to the present invention, the concentration of the drug on the balloon surface is differentiated along the axial extension of the balloon: in other words the concentration of applied drug is lower towards the portion with smaller diameter and it is higher towards the portion with higher diameter. In this way, the concentration of drug applied to the balloon compensates the asymmetrical geometry of the balloon along its axial extension. Therefore the drug concentration per mm2 balloon surface is constant over the balloon to reach homogeneous drug application to the tissue to be treated.

Moreover, the specific shape and dimension of the conical balloon according to the invention grants both a good dilation of the restenosis, since the balloon acts as a sort of wedge in order to expand the lumen of the blood vessel.

Moreover the use of lubricant helps the catheter reaching the target lesion without jamming within blood vessels and without damaging them during vessel cannulation.

A person skilled in the art may make numerous modifications and variations to the catheters described above so as to satisfy contingent and specific requirements, while remaining within the scope of protection of the invention as defined by the following claims.

1. A drug coated balloon catheter comprising
a connector,
a shaft extending from a proximal end to a distal end along an axial direction and having a guidewire lumen and an inflation lumen, the shaft being connected to the connector on said proximal end,
the shaft being provided with an inflatable balloon, fluidically connected with said inflation lumen in order to be selectively inflated and/or deflated, wherein
the balloon is conical and axis-symmetrical around a prevalent extension axis,
wherein the balloon has an increasing diameter, moving from a first end to a second end, said diameter being measured on a section plane perpendicular to said prevalent extension axis,
wherein an external surface of the balloon is coated with a drug for treating restenosis.

2. The drug coated balloon catheter according to claim 1, wherein the diameter of the balloon continuously increases moving from the first end to the second end.

3. The drug coated balloon catheter according to claim 1, wherein said first end faces the proximal end and said second end faces the distal end.

4. The drug coated balloon catheter according to claim 1, wherein said first end faces the distal end and said second end faces the proximal end.

5. The drug coated balloon catheter according to claim 1, wherein the diameter at the first end is 6 mm.

6. The drug coated balloon catheter according to claim 1, wherein the diameter at the second end is 7 mm.

7. The drug coated balloon catheter according to claims 1, wherein the diameter at the second end is 8 mm.

8. The drug coated balloon catheter according to claim 1, wherein the diameter at the second end is 9 mm.

9. The drug coated balloon catheter according to claim 1, wherein the length of the balloon or axial distance from the first end to the second end of the balloon is between 30 mm and 60 mm.

10. The drug coated balloon catheter according to claim 9, wherein said length of the balloon is about 45 mm.

11. The drug coated balloon catheter according to claim 1, wherein said conical balloon is configured so as to have a nominal or working pressure between 12 to 16 bar.

12. The drug coated balloon catheter according to claim 1, wherein said conical balloon is configured so as to have a burst pressure above 25 bar.

13. The drug coated balloon catheter according to claim 1, wherein said drug is a Paclitaxel solution.

14. The drug coated balloon catheter according to claim 1, wherein said drug on the external surface of the balloon has a concentration between 2 to 10 μg/mm2.

15. The drug coated balloon catheter according to claim 1, wherein said guidewire lumen is between 0.010 inches to 0.037 inches.

16. The drug coated balloon catheter according to claim 1, wherein the catheter has at least a dual-lumen construction provided with at least two lumens comprising said guidewire lumen and said inflation lumen, wherein said lumens are separated each other by an internal sept.

17. The drug coated balloon catheter according to claim 1, wherein an external wall of the shaft, opposite to said lumens, is covered with a lubricant.

18. The drug coated balloon catheter according to claim 17, wherein the entire external wall of the shaft is covered with a lubricant except for said balloon.

19. The drug coated balloon catheter according to claim 1, wherein the balloon has an increased wall thickness, comprised between 0.01 mm and 0.2 mm.

20. The drug coated balloon catheter according to claim 19, wherein such an increased wall thickness is obtained by a single sheath delimiting the balloon surface.

21. The drug coated balloon catheter according to claim 19, wherein such an increased wall thickness is obtained by the juxtaposition of two balloons.

22. The drug coated balloon catheter according to claim 1, wherein the balloon is stiffened by reinforcing fibers.

23. The drug coated balloon catheter according to claim 1, wherein the concentration of drug applied to the balloon surface is lower towards the portion with smaller diameter and it is higher towards the portion with higher diameter, so as to get a constant drug concentration over the balloon surface, along its axial extension.
